(19) United States
(12) Patent Application Publication
(10) Pub. No.: US 2007/0265541 A1
(43) Pub. Date: Nov. 15, 2007

(54) MP3 player capable of transmitting pulse data

(76) Inventor: Chin-Yeh Hung, Chang Pin Town (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/433,425

(22) Filed: May 15, 2006

(51) Int. Cl. A61B 5/04

(52) U.S. Cl. 600/523

(57) ABSTRACT

The present invention discloses a MP3 player capable of transmitting pulse data that includes a casing, a voltage detecting unit, two sets of left and right hand electrodes, a digital signal processor, a flash memory, an earphone, a plurality of input keys and a USB interface to achieve the functions of analyzing and evaluating a user's exercise and physiological conditions, in addition to the function of listening to music anywhere.

### BACKGROUND OF THE INVENTION

1. Field of the Invention

The invention relates to an application of a MP3 player, and more particularly to a MP3 player capable of measuring heartbeats, cumulatively recording the heartbeats in a flash memory, and using an Auto Run program and a USB interface to quickly and conveniently transmit the heartbeat data to a personal computer for performing analyses and evaluations, in addition to the function of playing music.

2. Description of the Related Art

The full name of MP3 is Movie Picture Experts Group (MPEG) 1 Layer 3 which belongs to a level of MPEG-1 with an original goal of reducing a load consumed in an information media transmission. The audio data of MP3 belongs to one of the acoustic wave data compressed into a MP3 1 format. The wave data of approximately 50 MB to 60 MB is compressed into 4 MB by this special compression method, and its tone quality is almost the same as that of CD music, but it provides almost ten times of the data storage capacity. Therefore, a MP3 music CD have a storage capacity of more than ten music CDs. If one music CD album can play 60 minutes of music, then one MP3 music CD can play several tens of hours of music data, and the MP3 player serves as a small music cabinet for providing users a portable music player, and thus the MP3 player and music CD become a new trend.

At present, more and more people listen to MP3 music while doing exercises (such as walking or jogging), and our heartbeats will change significantly before and after doing exercises, and the measurements of these variables during the exercises are very important. To maximize the benefits of the exercises, the exercisers must increase their heartbeat up to 60% of the maximum heartbeat rate. If the heartbeat rate rises up to its maximum during exercises, it will be harmful to our health, and thus a convenient and accurate heartbeat meter is necessary for our fitness and exercises.

There are various kinds of traditional fitness heartbeat measuring devices such as the pulse meters disclosed in U.S. Pat. Nos. 4,409,983, 4,224,948, 4,120,269 and 5,807,267. However, these traditional pulse meters adopt infrared detectors, exhaust sensors, piezo pressure sensors or optical sensors for detecting pulses, wherein the infrared sensors have less precision, since these sensors may be affected by the conditions of external environment easily.

In U.S. Pat. No. 5,807,267, a detector is installed at the bottom of a wrist band for detecting the heartbeat of a radial artery, but a piezo pressure sensor or an optical sensor is used as a sensor assembly to press closely to a radial artery for the detection, and such detecting method may be affected by the conditions of the external environment, and an accurate measuring position has a significant effect on the measured value. For medical professionals, they may use these instruments to detect accurate measurements, but for general athletes or consumers, they have difficulties to use such instruments to accurately measure the position. Furthermore, the exercisers have to carry both the MP3 and the wrist band with them for the application, which is very inconvenient, and traditional pulse detectors usually do not come with recording and analyzing functions and cannot be connected to a personal computer. The traditional pulse detectors cannot completely record and provide the change of heartbeat data of a user within several days, a week, or a month, and these pulse detectors cannot be used for analyzing and evaluating the physical conditions of the users. In summation, traditional pulse detectors require further improvements.

### SUMMARY OF THE INVENTION

Therefore, it is a primary objective of the present invention to provide a MP3 player capable of transmitting pulse data that skillfully integrates a MP3 player and a pulse detector for measuring heartbeats (or pulses) while listening to music anytime and anywhere.

Another objective of the present invention is to provide a MP3 player capable of transmitting pulse data that accumulates and stores the detected heartbeat and uses a built-in Auto Run program and a USB interface connected to a personal computer to automatically send the heartbeat recorded in a certain period of time to the personal computer for analyses and evaluations and further provide correct personal health data of the users.

A further objective of the present invention is to record the heartbeats in the personal computer and send the heartbeat data to a doctor's personal computer at a remote end through the Internet and a platform server to achieve the effect of further evaluations and bidirectional communications.

In order to achieve the above-mentioned objectives, a MP3 player capable of transmitting pulse data includes:
a) a casing made of a non-conducting material and having an LCD display device disposed at the front of the casing;
b) a voltage detecting unit installed in the casing for detecting a voltage frequency change of a human body caused by blood circulation;
c) a set of right hand electrode having a first conductive rubber and a second conductive rubber respectively disposed at an upper surface and a lower surface on the right side of the casing, and electrically coupled to the voltage detecting unit in the casing for touching and pressing two fingers of a user's right hand;
d) a set of left hand electrode having a third conductive rubber and a fourth conductive rubber respectively disposed at an upper surface and a lower surface on the left side of the casing, and electrically coupled to the voltage detecting unit in the casing for touching and pressing two fingers of a user's left hand, and the third conducing rubber being coupled with the second conductive rubber to form a common contact point wherein the left and right hand electrodes are coupled separately to three inductors of the voltage detecting unit for sending a measured voltage frequency change after going through an electrostatic charge filter to a filtering and shaping and amplifying circuit to obtain a signal;
e) a digital signal processor for receiving a signal transmitted by the voltage detecting unit, and accumulating the signal to obtain a user's pulse (heartbeat) value;
f) a flash memory having a partitioned memory block with at least one area for storing an Auto Run program and another area for being used as general memories for storing an audio data and producing an audio signal after decoding by the digital signal processor;
g) an earphone for converting an audio signal outputted from the digital signal processor into a sound;
h) a power supply unit installed in the casing for supplying electric power required by each of the foregoing elements;
i) a plurality of input keys disposed at the surface of the casing for users to operate and send an instruction to the digital signal processor; and
j) a USB interface installed at a side of the casing and electrically coupled to the digital signal processor, such that when the USB interface is connected to a personal computer, an Auto Run program pre-stored in the flash memory can automatically send the pulse data accumulated in a predetermined time by a user to an operating system of the personal computer, so as to facilitate recording and analyzing a user's physiological conditions.

### BRIEF DESCRIPTION OF THE FIGS.

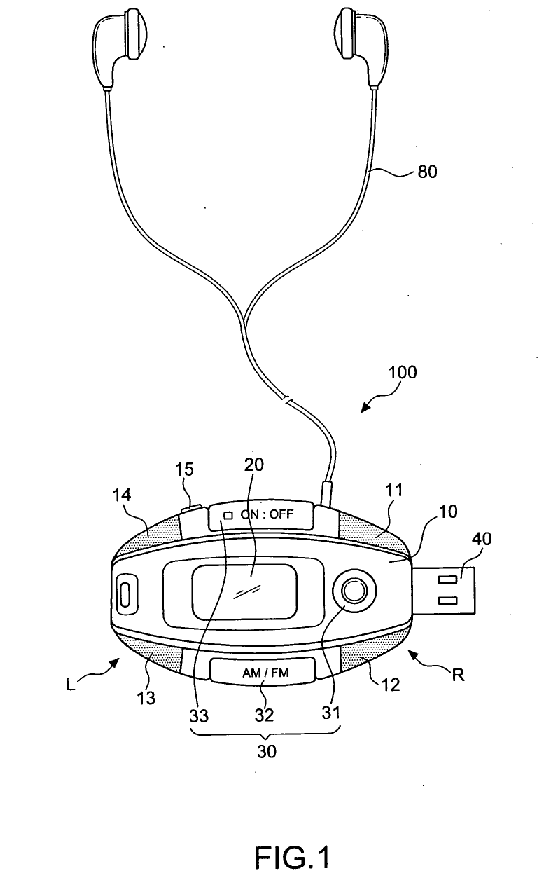

FIG. 1 is a schematic view of a preferred embodiment of the invention;

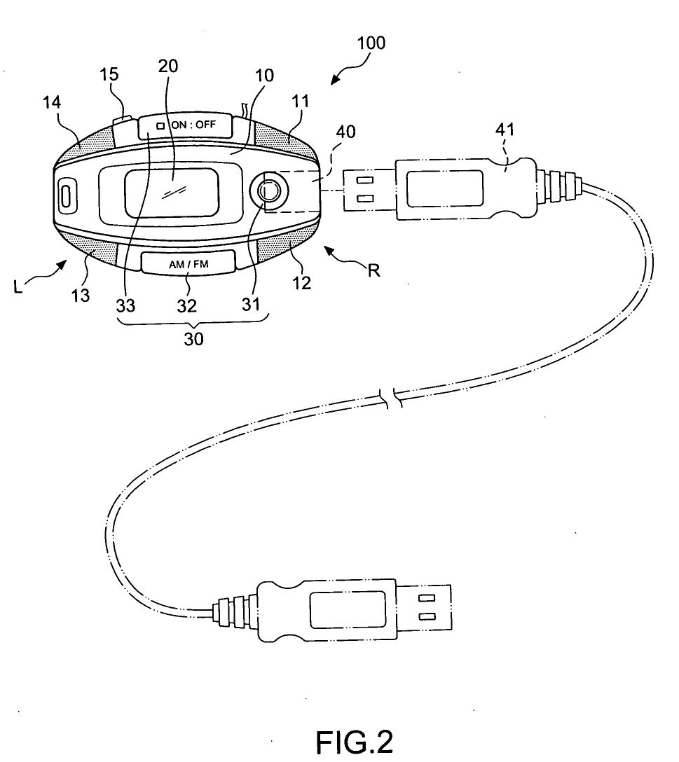

FIG. 2 is a schematic view of connecting a USB connector with the invention;

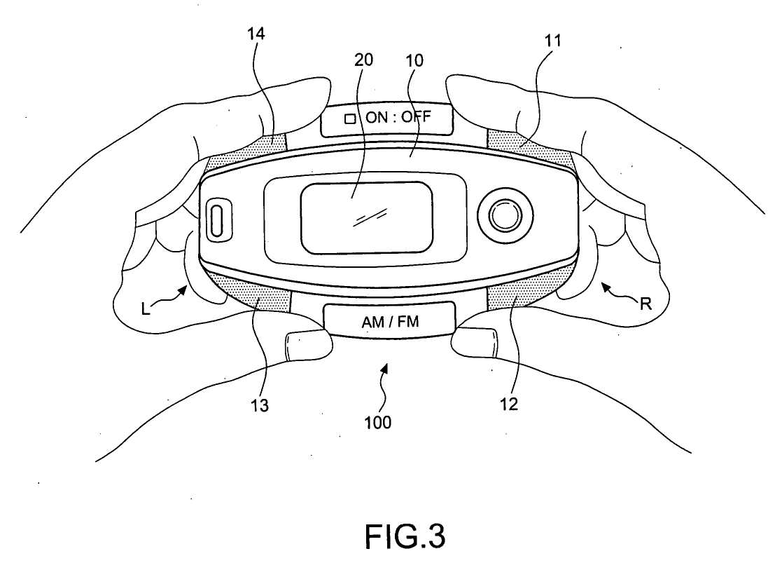

FIG. 3 is a schematic view of measuring heartbeats according to the invention;

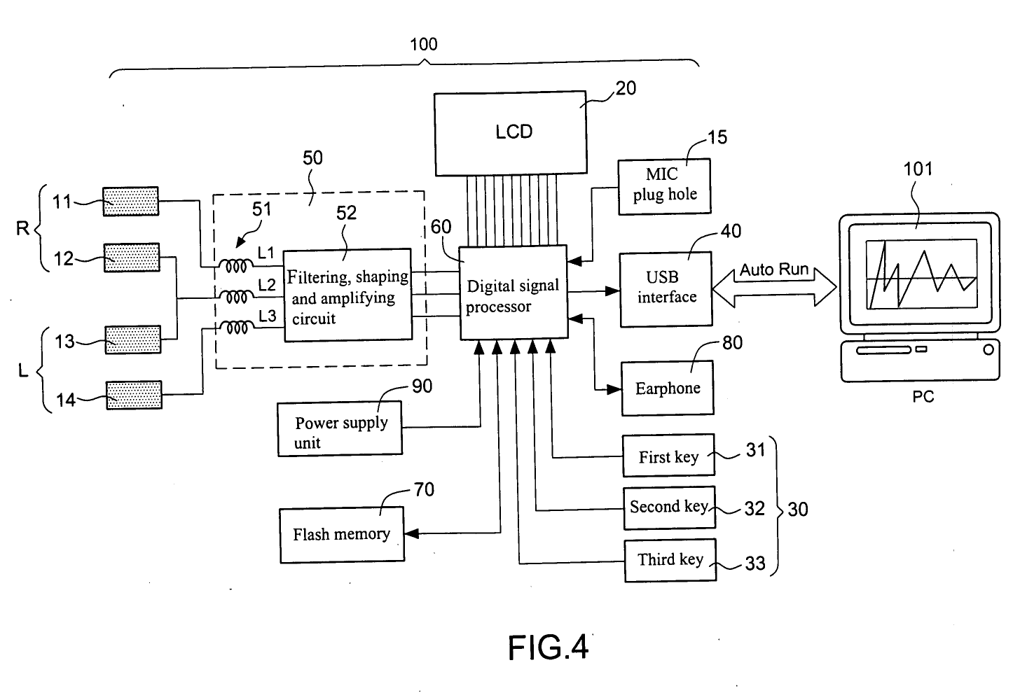

FIG. 4 is a circuit block diagram of the invention;

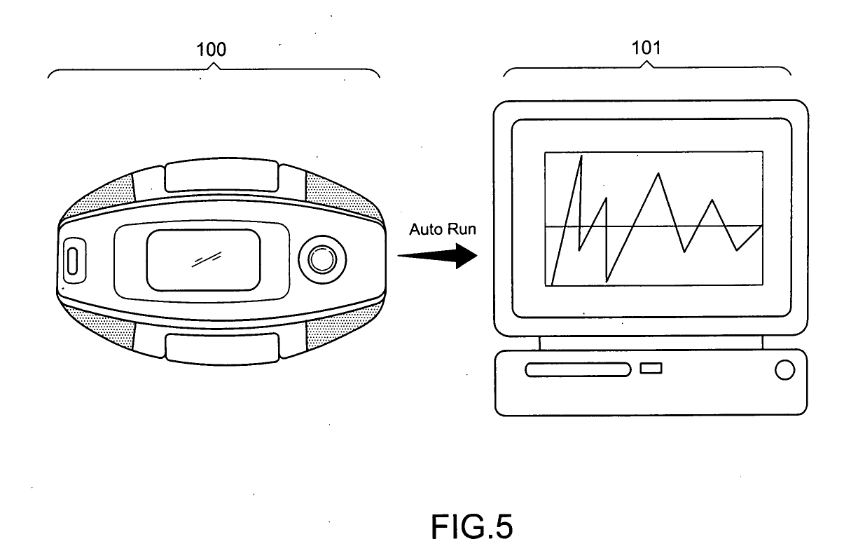

FIG. 5 is a schematic view of connecting a computer with the invention;

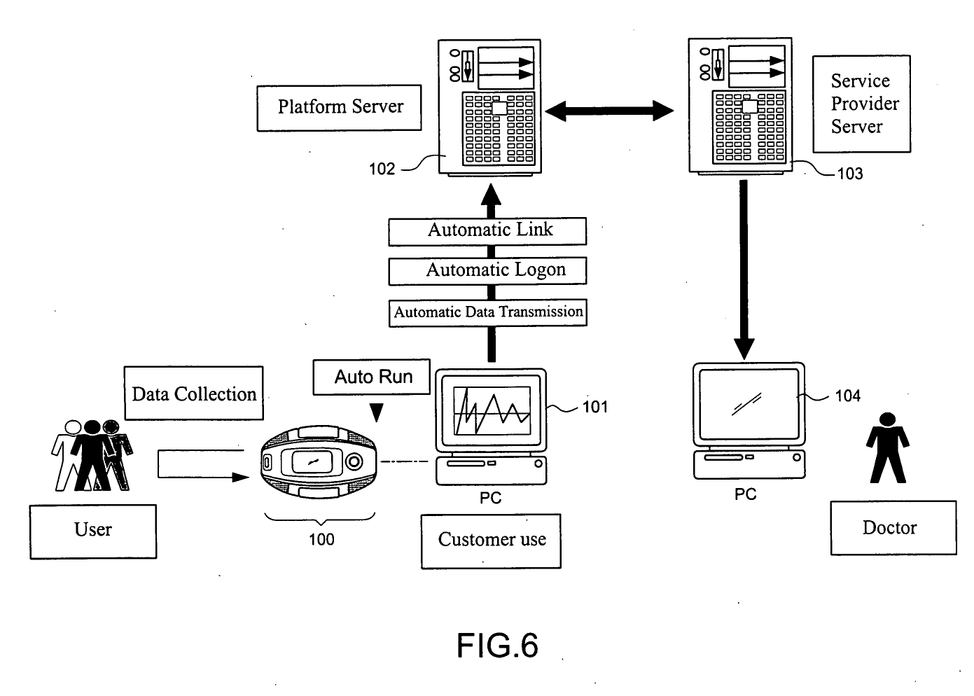

FIG. 6 is a schematic view of an application of the invention; and

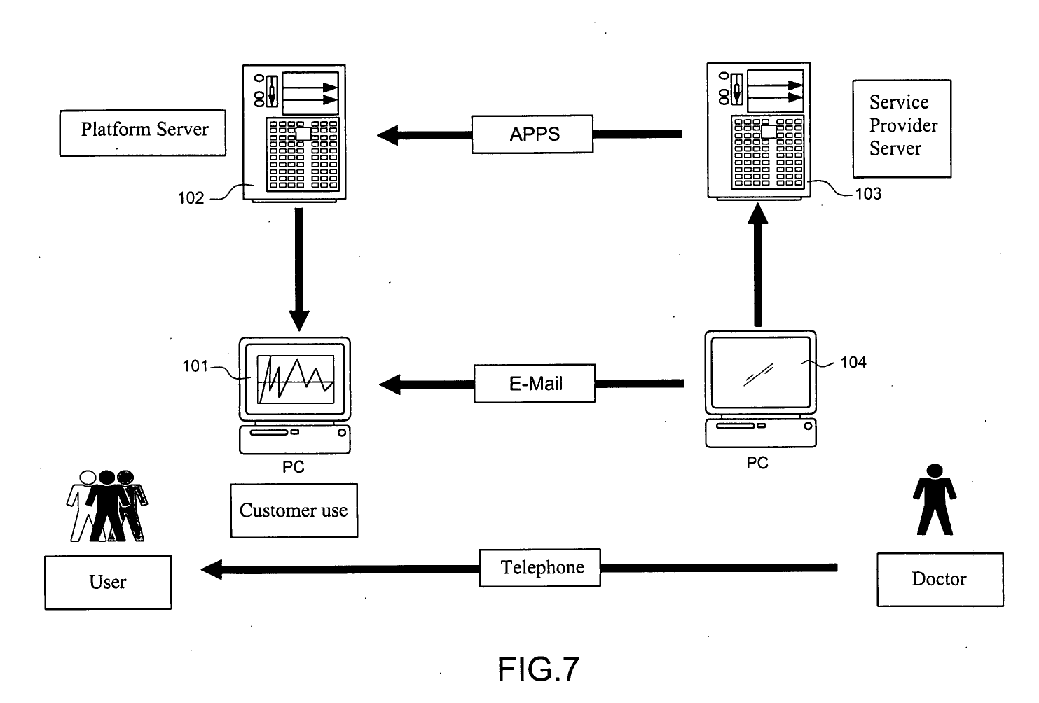

FIG. 7 is a schematic view of another application of the invention.

### DETAILED DESCRIPTION OF THE PREFERRED EMBODIMENT

Referring to FIGS. 1 to 5, a pulse MP3 player 100 according to a preferred embodiment of the present invention comprises the following elements:

A casing 10 is made of a non-conducting material and comes with the size of a general MP3 player and includes an LCD display device 20 disposed at the front of the casing 10.

A voltage detecting unit 50 is installed in the casing 10 for detecting a voltage frequency change caused by the blood circulation of a human body, and the voltage detecting unit 50 comprises a plurality of inductors 51 and a filter shaping and amplifying circuit 52 as shown in FIG. 4.

A set of right hand electrode (R) comprises a first conductive rubber 11 and a second conductive rubber 12 respectively installed at the upper and lower surfaces of the right side of the casing 10 and electrically coupled to the voltage detecting unit 50 in the casing 10 for pressing and touching two fingers of a user's right hand.

Similarly, a set of left hand electrode (L) comprises a third conductive rubber 13 and a four conductive rubber 14 respectively installed at the upper and lower surface of the left side of the casing 10 and electrically coupled to the voltage detecting unit 50 in the casing 10 for pressing and touching two fingers of a user's left hand as shown in FIG. 3. Since the casing 10 is an insulator made of a non-conducting material, therefore the foregoing four conductive rubbers 11, 12, 13, 14 are installed at the edges of the casing 10 for facilitating the pressing and touching of the user's fingers in addition to the function of separating the electrodes, and thus the heartbeat of the user can be measured under the best condition of naturally touching the fingers. In a preferred embodiment as shown in FIG. 4, the first conductive rubber 11 is coupled to the first inductor L1, and the second and third conductive rubbers 12, 13 have a common contact point coupled to the second inductor L2. The fourth conductive rubber 14 is coupled to the third inductor L3, so that the foregoing four conductive rubbers 11, 12, 13, 14 constitute the electrodes for pressing and touching the right and left hands. Since the conductive rubber is similar to metals having a good conductivity and a frictional anti-slippery texture, therefore when the four fingers of the left and right hand press and touch the four conductive rubbers 11, 12, 13, 14 as shown in FIG. 3, no slippery will occur even if there are sweats on the fingers. Thus, an accurate voltage frequency change caused by blood circulations can be detected accurately. If the four fingers of the user's left and right hands touch the four conductive rubbers 11, 12, 13, 14 simultaneously, the left and right sets of electrodes (L), (R) will be connected electrically, and a voltage will be produced in the blood circulation of a human body. If the heartbeat rate varies, the frequency of a voltage change will be different. The farther the blood in a human body, the more significant is the change of the voltage frequency. The present invention makes use of the left and right hands that are touched with different electrodes, and thus the blood circulates the farthest between the left and right hands, and the most significant frequency of voltage change of the two sets of electrodes (L), (R) can be detected to obtain a more accurate measurement of the heartbeat.

In FIG. 4, two sets of left and right electrodes (L), (R) send the measured voltage change frequency to the filtering, shaping and amplifying circuit 52 after going through the electrostatic filter of the inductor 51 to obtain a shaped and amplified signal. However, such circuit 52 is a prior art, and thus will not be described here.

A digital signal processor (DSP) 60 includes a MCU featuring high speed, high bit rate, data index shift command, and programmable digital signal processing capabilities for the voice, communication, image processing, and data control applications. In this preferred embodiment, the digital signal processor 60 can execute a program stored in a flash memory 70 for decoding a MP3 file or a compressed audio file in the control of playback operations to produce corresponding audio signals and provides a general MP3 playback function. Further, the digital signal processor 60 can receive the signal sent from the foregoing voltage detecting unit 50, and the signals are accumulated to obtain a user's heartbeats (or pulses). Of course, such heartbeat can be displayed on the LCD display device 20.

Since the present pulse detectors such as the aforementioned wrist band can detect heartbeats only, but cannot perform further recording and analysis, therefore such pulse detectors cannot provide the information about the pulse change of a user within a week or a month and it is difficult to evaluate a user's physiological conditions and health.

To overcome the foregoing shortcomings, the present invention uses a flash memory 70 as a memory unit that includes partitioned memory blocks with at least one area for storing an Auto Run program and other areas used as general memories, and the flash memory 70 can store audio data and produce audio signals after the data are decoded and processed by the digital signal processor 60.

In FIG. 1, the present invention further comprises an earphone 80 for converting audio signals outputted by the digital signal processor 60 into sound. A plurality of input keys 30 are installed at a surface of the casing 10 for users to operate and send a command to the digital signal processor 60. For example, a first key 31 is used for the ON/OFF/Play/Pause functions, and a second key 32 is used as the direction keys, and the third key 33 is used for the Recording/FM functions. However, these keys can be designed as needed, but not limited to the abovementioned applications.

In FIG. 4, a power supply unit 90 is installed in the casing 10 for supplying electric power to the foregoing components, and the power supply unit 90 could be an AAA battery or a lithium battery.

A USB interface 40 is installed at a side of the casing 10 as shown in FIG. 1 and substantially in the form of a male connector protruded from a side of the casing 10. Of course, the USB interface 40 also could be in the form of a female connector concavely disposed in the casing 10 as shown in FIG. 2, and a USB cable 41 is used for the connection.

In FIG. 4, the USB interface 40 is electrically coupled to the digital signal processor 60. If the USB interface 40 is connected to a personal computer 101, the Auto Run program pre-stored in the flash memory 70 will automatically send the pulse data measured and accumulated in a period of time such as a week, a month, or a year to the personal computer 101, so that the original hardware, software and operating system of the personal computer 101 can be applied as shown in FIG. 5. If the pulse MP3 player 100 of the present invention is connected to a personal computer 101, the pulse data can be transmitted automatically, and thus the application is very convenient, and users can obtain the pulse change in a predetermined period, and graphically analyze and evaluate the data to understand their physical conditions.

In addition, the casing 10 includes a microphone (MIC) plug hole 15 disposed at an appropriate position, but the plug hole 15 is an optional item and not a necessary item.

The present invention makes use of the foregoing technical measures for the following improvements:

1. The invention uses two fingers of each of a user's left and right hands to press and touch the electrodes (L), (R) of the four conductive rubbers 11, 12, 13, 14 of the pulse MP3 player 100, and thus providing a convenient application and a good contact for accurately measuring heartbeats.

2. The present invention uses the Auto Run program stored in the flash memory 70, such that when the pulse MP3 player 100 is connected to the personal computer 101, all heartbeat data measured in a certain period of time will be displayed on the personal computer 101 immediately to facilitate users to understand their physiological conditions. Further, the data collected by the users can be obtained as shown in FIG. 6, and the personal computer 101 automatically sends data, logs on, and links to a platform server 102 which is connected to a service provider server 103, such that a doctor at a remote end can obtain the user's physiological information through the personal computer 104, so as to perform a periodical tracking and give medical advices. In FIG. 7, the doctor can analyze and evaluate the conditions and inform the users through a telephone or an E-mail or the data can be uploaded to the service provider server 103, so that the users can obtain the doctor's analysis reports and medical advices from their personal computer 101 through the platform server 102, and its application is very convenient and practical.

Many changes and modifications in the above-described embodiment of the invention can, of course, be carried out without departing from the scope thereof. Accordingly, to promote the progress in science and the useful arts, the invention is disclosed and is intended to be limited only by the scope of the appended claims.

What is claimed is:

1. A MP3 player capable of transmitting pulse data, comprising:
a) a casing made of a non-conducting material and having an LCD display device disposed at the front of the casing;
b) a voltage detecting unit installed in the casing for detecting a voltage frequency change of a human body caused by blood circulation;
c) a set of right hand electrode having a first conductive rubber and a second conductive rubber respectively disposed at an upper surface and a lower surface on the right side of the casing, and electrically coupled to the voltage detecting unit in the casing for touching and pressing two fingers of a user's right hand;
d) a set of left hand electrode having a third conductive rubber and a fourth conductive rubber respectively disposed at an upper surface and a lower surface on the left side of the casing, and electrically coupled to the voltage detecting unit in the casing for touching and pressing two fingers of a user's left hand, and the third conducing rubber being coupled with the second conductive rubber to form a common contact point wherein the left and right hand electrodes are coupled separately to three inductors of the voltage detecting unit for sending a measured voltage frequency change after going through an electrostatic charge filter to a filtering and shaping and amplifying circuit to obtain a signal;
e) a digital signal processor for receiving a signal transmitted by the voltage detecting unit, and accumulating the signal to obtain a user's pulse (heartbeat) value;
f) a flash memory having a partitioned memory block with at least one area for storing an Auto Run program and another area for being used as general memories for storing an audio data and producing an audio signal after decoding by the digital signal processor;
g) an earphone for converting an audio signal outputted from the digital signal processor into a sound;
h) a power supply unit installed in the casing for supplying electric power required by each of the foregoing elements;
i) a plurality of input keys disposed at the surface of the casing for users to operate and send an instruction to the digital signal processor; and
j) a USB interface installed at a side of the casing and electrically coupled to the digital signal processor, such that when the USB interface is connected to a personal computer, an Auto Run program pre-stored in the flash memory can automatically send the pulse data accumulated in a predetermined time by a user to an operating system of the personal computer, so as to facilitate recording and analyzing a user's physiological conditions.

2. The MP3 player capable of transmitting pulse data as recited in claim 1, wherein the casing further comprises a microphone (MIC) plug hole.

3. The MP3 player capable of transmitting pulse data as recited in claim 1, wherein the USB interface is in a form of a male connector protruded from a side of the casing.

4. The MP3 player capable of transmitting pulse data as recited in claim 1, wherein the USB interface is in a form of a female connector concavely disposed on a side of the casing.

5. The MP3 player capable of transmitting pulse data as recited in claim 1, wherein the power supply unit includes a battery.
